(19) United States
(12) Patent Application Publication
(10) Pub. No.: US 2023/0355540 A1
(43) Pub. Date: Nov. 9, 2023

(54) STROKE TREATMENT

(71) Applicants: Oxford University Innovation Limited, Oxford (GB); PRESIDENT AND FELLOWS OF HARVARD COLLEGE, Cambridge, MA (US)

(72) Inventors: Daniel Beard, Belmont (AU); Donald Ingber, Boston, MA (US); Oktay Uzun, Boston, MA (US); Frank Bobe, Harvard, MA (US)

(21) Appl. No.: 18/029,028

(22) PCT Filed: Sep. 28, 2021
(86) PCT No.: PCT/US2021/052402

§ 371 Date: Mar. 28, 2023

(30) Foreign Application Priority Data
Sep. 29, 2020 (GB) 2015386.2
May 19, 2021 (GB) 2107137.8

(51) Int. Cl. A61K 9/51; A61K 45/06; A61K 9/00; A61K 31/4418; A61K 31/21; A61P 9/10

(52) U.S. Cl. CPC A61K 9/5153; A61K 45/06; A61K 9/0019; A61K 31/4418; A61K 31/21; A61P 9/10; B82Y 5/00

(57) ABSTRACT

The present invention relates to a shear-activated nanotherapeutic (SA-NT) for use in treating stroke by increasing blood supply to the brain via collateral vessels, wherein the SA-NT comprises an aggregate comprising a plurality of nanoparticles, the aggregate further comprising one or more vasodilating agents or pharmaceutically acceptable salts thereof; wherein the aggregate is configured to disaggregate above a predetermined shear stress.

### FIELD

The present invention relates to the treatment of ischaemic stroke by increasing blood flow in collateral vessels supplying the penumbra.

### BACKGROUND TO THE INVENTION

Stroke is a major health problem worldwide. Recent estimations (Feigin V L et al. Global and regional burden of stroke during 1990-2010: findings from the Global Burden of Disease Study 2010. Lancet. 2014; 383(9913):245-254) indicate that the global prevalence rate is 5 strokes per 1000 person-years, corresponding to 33 million people living after a stroke. 5.9 million people suffered a stroke-related death in 2010, and stroke resulted in more than 102 million lost disability-adjusted life years (DALYs), corresponding to the sum of premature death and years of healthy life lost attributed to disability. In recent years, little progress has been made in stroke therapy options or in reducing stroke-related mortality/morbidity rates.

Stroke occurs when there is an obstruction of blood flow to the brain by a thrombus or a clot. The ensuing reduction in blood supply causes brain cell damage and death. Ischaemic stroke occurs when the blood supply to the brain is either completely cut off by the thrombus, or is severely reduced. This differs from a haemorrhagic stroke which occurs when blood from an artery bleeds into the brain due to damage or bursting of a blood vessel.

Currently, there is no specific therapy for ischaemic stroke other than stopping the ischaemic insult via thrombectomy or thrombolysis. Minimising the time the brain is deprived of adequate blood supply due to the thrombus or clot is key to salvaging ischaemic tissue.

The reduction in blood flow and rate of cell death following stroke is not uniform. In the central territory (the ischaemic core) blood flow reduction is severe and results in rapid tissue death. Surrounding the core, there may be sufficient residual perfusion to keep the brain tissue alive for a limited amount of time. The tissue in this surrounding area (the ischaemic penumbra) can be saved if the occluded artery is opened quickly enough. After the onset of an ischaemic stroke, the penumbra receives its residual perfusion from the adjoining non-obstructed arterial territories via leptomeningeal collateral (or bypass) blood vessels (also referred to herein as “collaterals” or “collateral vessels”) on the surface of the brain. Clinical studies have shown that greater flow through these vessels is associated with a larger penumbra (smaller core region of cell death) and better the outcome for stroke patients. For instance, Vagal et al. (Stroke (2018) 49(9): 2102-2107) reports lower penumbral salvage for stroke patients with poor collaterals than those with robust collaterals. Wufuer et al. (Exp Ther Med. 2018 January; 15(1):707-718) reports improved outcomes for stroke patients with good collateral circulation status following thrombolysis treatment, while Leng et al. (J Neurol Neurosurg Psychiatry. 2016 May; 87(5):537-44) reports improved outcomes for stroke patients with good pre-treatment collateral status following endovascular (thrombectomy) treatment.

Therapies that can maintain or enhance blood flow through collateral blood vessels have the potential to keep cells in the ischaemic penumbra alive for longer, extending the window in which the occluded cerebral artery could be opened. Despite the proven importance of collateral vessel status in human stroke outcome, however, there is little existing research into such therapies targeting collateral blood vessels. As such, collateral therapeutics is a nascent field.

Previously suggested collateral therapeutic strategies include interventions that increase collateral perfusion, e.g. by increasing blood pressure or by vasodilation. These approaches are largely based around either enhancing the driving pressure of blood to the brain or dilating collateral vessels. Although a number of these interventions have been shown to improve collateral flow in animal models of stroke (e.g. induced hypertension, partial aortic occlusion, inhaled nitric oxide and sphenopalatine ganglion stimulation) their ultimate clinical utility is limited due to issues surrounding invasiveness, side effects and the need for specialist equipment. For example, increasing blood pressure may easily lead to haemorrhage or rupture of blood vessels in an already compromised patient. Further, vasodilation therapies are likely to dilate vascular beds elsewhere in the brain and body. This leads to vascular steal (i.e. dilation of the peripheral vascular network “steals” blood flow from another region, such as the brain), resulting in drops in systemic perfusion pressure ultimately making stroke outcome worse.

For example, it has recently been shown (Bath et al., The Lancet, Volume 393, Issue 10175, pages 1009-1020) that transdermal (i.e. systemic) delivery of nitroglycerin, a potent vasodilating agent, did not improve functional outcome in patients suffering ultra-acute stroke.

There is therefore a need for further therapies that improve outcomes for stroke patients.

### SUMMARY OF THE INVENTION

The present invention describes a strategy for selectively dilating collateral vessels while reducing or avoiding the deleterious side effects associated with systemic blood vessel dilation during ischaemic stroke. Accordingly, the present invention relates to a shear-activated nanotherapeutic (SA-NT) for use in treating stroke by increasing blood supply to the brain via collateral vessels, wherein the SA-NT comprises an aggregate comprising a plurality of nanoparticles (a nanoparticle aggregate/NPA), the nanoparticle aggregate further comprising one or more vasodilating agents or pharmaceutically acceptable salts thereof; wherein the nanoparticle aggregate is configured to disaggregate above a predetermined shear stress.

The present invention also relates to a composition for use in treating stroke by increasing blood supply to the brain via collateral vessels, wherein the composition comprises such a SA-NT in combination with one or more pharmaceutically acceptable excipients, carriers and/or diluents.

The present invention also relates to a method of treating stroke by increasing blood supply to the brain via collateral vessels comprising administering such a SA-NT or such a composition to a patient in need thereof.

The present invention also relates to the use of such a SA-NT or such a composition for the manufacture of a medicament for treating stroke by increasing blood supply to the brain via collateral vessels.

The present invention also relates to a SA-NT comprising an aggregate comprising a plurality of nanoparticles; wherein the aggregate is configured to disaggregate above a predetermined shear stress; and wherein the aggregate further comprises from about 0.1% to about 20% by weight of one or more vasodilating agents. Preferably, the aggregate comprises from about 0.4% to about 2.5% by weight of nitroglycerin, or a pharmaceutically acceptable salt thereof, as the one or more vasodilating agents.

The use of a SA-NT as a collateral therapeutic has a number of distinct advantages over previously-trialled collateral therapeutics, which may enhance clinical utility. For example, SA-NTs are easily administered directly into the blood stream, for example intravenously. There is no need for specialist equipment and the treatment is non-invasive. The treatment may be administered upon arrival at hospital, or prior to arrival in hospital, for example by a paramedic responding to an emergency. It is therefore an improved method to increase blood flow to the brain following a stroke without reducing systemic blood pressure.

Selective treatment by increasing blood flow to the collateral vessels, by collateral vessel dilation, also has advantages over previous treatments which target the occlusion causing the stroke itself. In particular, administration of drugs targeting the occlusion itself is delayed until diagnosis of the nature of the stroke. The present invention, which specifically targets the collateral vessels, therefore also has advantages over such treatments as it can be administered immediately following the stroke, as an emergency therapy. This maximises the survival of brain tissue in the period between occurrence of the stroke and administration of further therapies.

Additionally, SA-NTs are only activated at the site of the collateral vessels releasing the vasodilator. This minimizes systemic exposure to the vasodilator and improves the vasodilatory effect of the vasodilator.

### BRIEF DESCRIPTION OF THE DRAWINGS

FIG. 1 shows the effect of injection of SA-NTs comprising nitroglycerin-containing nanoparticle aggregates (NG-NPAs) and blank NPAs on collateral perfusion in a rat model of stroke.

FIG. 2 shows average change in collateral perfusion during treatment with NG-NPAs and blank NPAs in a rat model of stroke.

FIG. 3 shows peak collateral perfusion obtained using NG-NPAs compared to increasing doses of “free” nitroglycerin (i.e. soluble nitroglycerin free of NPAs) in a rat model of stroke.

FIG. 4 shows collateral perfusion and mean arterial pressure upon administration of an infusion of NG-NPAs at a rate of 2.75 μg/kg/min in a rat model of stroke.

FIG. 5 shows collateral perfusion and mean arterial pressure upon administration of an infusion of NG-NPAs at a rate of 4.15 μg/kg/min in a rat model of stroke.

FIG. 6 shows changes in collateral perfusion as a function of time following administration of an infusion of NG-NPAs at a rate of 4 μg/kg/min to a first group and administration of an infusion of blank NPAs to a second group in a rat model of stroke.

FIG. 7 shows changes in perfusion in the contralateral/control hemisphere as a function of time following administration of an infusion of NG-NPAs at a rate of 4 μg/kg/min to a first group and administration of an infusion of blank NPAs to a second group in a rat model of stroke.

FIG. 8 shows changes in mean arterial pressure as a function of time following administration of an infusion of NG-NPAs at a rate of 4 μg/kg/min to a first group and administration of an infusion of blank NPAs to a second group in a rat model of stroke.

FIG. 9 shows infarct size following treatment with NG-NPAs and blank NPAs in a rat model of stroke.

FIG. 10 shows the relationship between collateral perfusion and infarct volume in a rat model of stroke (including both NG-NPA and blank NPA groups).

FIG. 11 shows changes in collateral perfusion as a function of time following administration of an infusion of free nitroglycerin (GTN) at a rate of 4 μg/kg/min to a first group and administration of an infusion of saline to a second group in a rat model of stroke.

FIG. 12 shows changes in perfusion in the contralateral/control hemisphere as a function of time following administration of an infusion of free nitroglycerin (GTN) at a rate of 4 μg/kg/min to a first group and administration of an infusion of saline to a second group in a rat model of stroke.

FIG. 13 shows changes in mean arterial pressure as a function of time following administration of an infusion of free nitroglycerin (GTN) at a rate of 4 μg/kg/min to a first group and administration of an infusion of saline to a second group in a rat model of stroke.

FIG. 14 shows changes in collateral perfusion as a function of time following administration of an infusion of free nitroglycerin (GTN) at a rate of 40 μg/kg/min to a first group and administration of an infusion of saline to a second group in a rat model of stroke.

FIG. 15 shows changes in perfusion in the contralateral/control hemisphere as a function of time following administration of an infusion of free nitroglycerin (GTN) at a rate of 40 μg/kg/min to a first group and administration of an infusion of saline to a second group in a rat model of stroke.

FIG. 16 shows changes in mean arterial pressure as a function of time following administration of an infusion of free nitroglycerin (GTN) at a rate of 40 μg/kg/min to a first group and administration of an infusion of saline to a second group in a rat model of stroke.

### DETAILED DESCRIPTION OF THE INVENTION

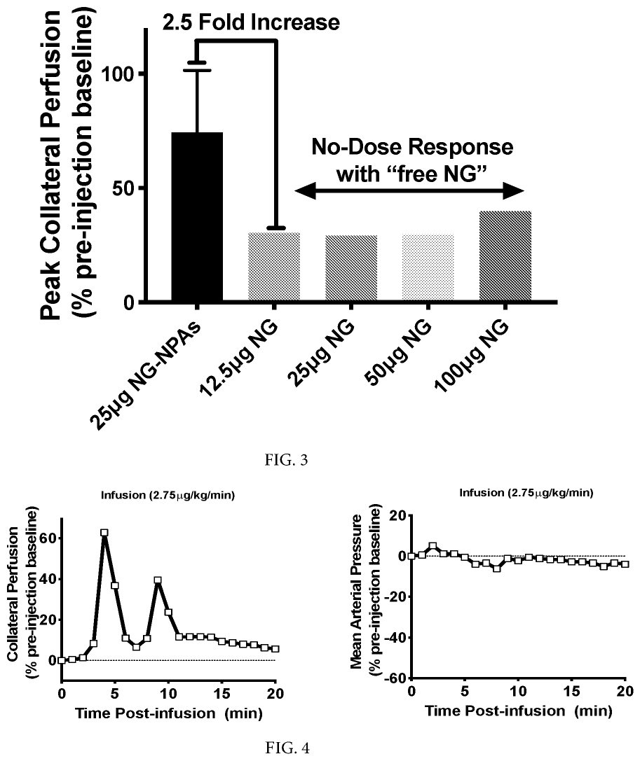

SA-NTs comprise aggregates which are similar in size to natural platelets, but are fabricated as aggregates of smaller nanoparticles. These aggregates remain intact when flowing in blood under normal physiological flow conditions. However, when exposed to shear stress elevated above normal levels (>100 dyne/cm²), the aggregates break up into individual nanoparticles. These nanoparticles experience lower drag forces than the larger aggregate particles, and adhere more efficiently to the surface of the blood vessels through which they are flowing. Any therapeutic agent carried by the nanoparticles may thus be selectively delivered to the site of disaggregation in regions of localised high shear stress. Previously, SA-NTs have been used to deliver the thrombolytic agent tissue plasminogen activator (t-PA) in thromboembolic disorders such as pulmonary embolism (see, for example, FIG. 3 of Korin et al. (Science. 2012 Aug. 10; 337(6095):738-42). However, the disclosure of Korin is restricted to the release of therapeutic agents in occluded vessels. Targeting non-occluded vessels that experience abnormally high shear stress due to abnormally high flow has not previously been considered.

During stroke, the blood flow velocity in the collateral vessels is extremely high due to the large pressure difference between the two vascular territories that the collateral vessels connect (healthy vascularised tissue and the penumbral tissue). This significantly raises shear stress, independent of vessel diameter changes (see, for example, Beard, D. J. et al. (2015). “Intracranial Pressure Elevation Reduces Flow through Collateral Vessels and the Penetrating Arterioles they Supply, a Possible Explanation for ‘Collateral Failure’ and Infarct Expansion after Ischemic Stroke. Journal of Cerebral Blood Flow & Metabolism”, 35(5), 861-872).

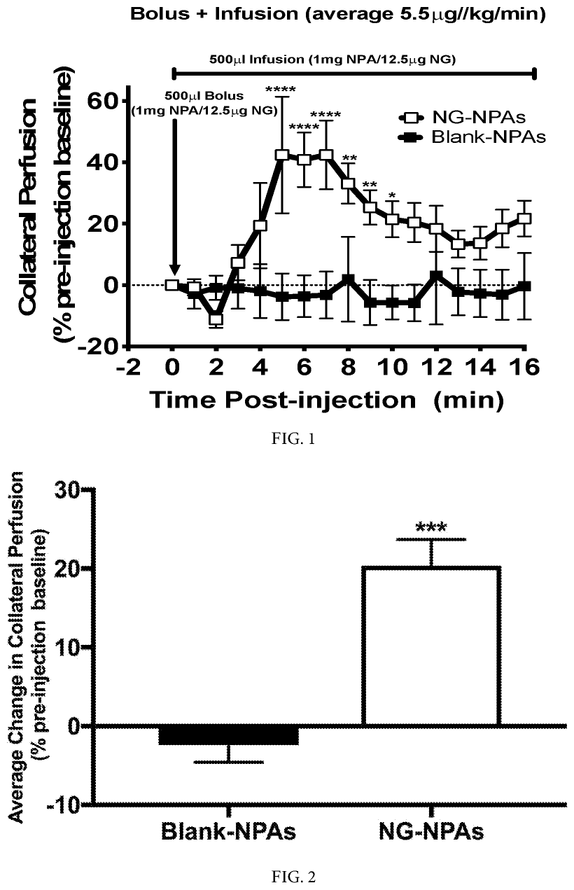

Despite the fact that the collateral vessels are not occluded following stroke, the present inventors have found that selective delivery to these vessels can be achieved by administration of a SA-NT. As demonstrated by the Examples and corresponding FIGS. 1 to 5, such a SA-NT provides a controllable and effective way of artificially enhancing perfusion of collateral vessels following stroke. This increases the blood flow to the cells of the penumbra, and enhances brain survival in stroke patients in which the clot remains in place.

The present invention provides a readily accessible therapy which may be used in centres without access to clot retrieval therapy (for example, in smaller emergency treatment centres). This novel therapy extends the window in which the cells of the penumbra can remain alive. This is not only of direct benefit in increasing the recovery of brain tissue following stroke, but may, for example, provide additional time for the patient to be transported to a larger specialist centre for clot retrieval, maximising the likelihood of the patient achieving a favourable clinical outcome.

### Nanoparticles

The SA-NTs described herein comprise aggregates of nanoparticles. In the present invention, the nanoparticles are of the order of from about 1 nm to about 1000 nm in size. Typically, the average diameter of the nanoparticles is from about 50 nm to about 500 nm, preferably from about 70 nm to about 400 nm, preferably from about 90 nm to about 300 nm, and preferably from about 100 nm to about 240 nm.

Nanoparticles suitable for drug delivery are well known in the art. Accordingly, nanoparticles used in the SA-NT of the present invention may include those described in, for example, Korin et al. (Science. 2012 Aug. 10; 337(6095):738-42), U.S. Pat. Nos. 6,645,517; 5,543,158; 7,348,026; 7,265,090; 7,541,046; 5,578,325; 7,371,738; 7,651,770; 9,801,189; 7,329,638; 7,601,331; 5,962,566; U.S. Pat. App. Pub. No. US2006/0280798; No. 2005/0281884; No. US2003/0223938; 2004/0001872; No. 2008/0019908; No. 2007/0269380; No. 2007/0264199 No. 2008/0138430; No. 2005/0003014; No. 2006/0127467; No. 2006/0078624; No. 2007/0243259 No. 2005/0058603; No. 2007/0053870; No. 2006/0 105049; No. 2007/0224277; No. 2003/0147966 No. 2003/0082237; No. 2009/0226525; No. 2006/0233883; No. 2008/0193547; No. 2007/0292524 No. 2007/0014804; No. 2004/02 19221; No. 2006/0193787; No. 2004/0081688; No. 2008/0095856; No. 2006/0134209; No. 2004/0247683 and WO 2013/185032, the content of all of which is incorporated herein by reference. Particularly suitable examples of nanoparticles for use in the present invention are those described in Korin et al. (Science. 2012 Aug. 10; 337(6095):738-42) and WO 2013/185032, the content of which is incorporated herein by reference.

For example, types of nanoparticles that can be used in forming the aggregates described herein may be: (1) nanoparticles formed from a polymer or other material to which one or more vasodilating agents absorbs/adsorbs or forms a coating on a nanoparticle core; (2) nanoparticles formed from a core formed by one or more vasodilating agents, which is coated with a polymer or other material; (3) nanoparticles formed from a polymer or other material to which one or more vasodilating agents is covalently linked; (4) nanoparticles formed from one or more vasodilating agents and other molecules; (5) nanoparticles formed so as to comprise a generally homogeneous mixture of one or more vasodilating agents with a constituent of the nanoparticle or other non-drug substance; (6) nanoparticles of pure drug or drug mixtures with a coating over a core of one or more vasodilating agents; (7) nanoparticles without any associated vasodilating agents; (8) nanoparticles composed entirely of one or more vasodilating agents; (9) nanoparticles which have one or more vasodilating agents permeated in the nanoparticles; and (10) nanoparticles which have one or more vasodilating agents adsorbed to the nanoparticles.

The nanoparticles typically comprise one or more biocompatible polymers. The biocompatible polymers may be biodegradable or non-biodegradable, but are preferably biodegradable. In some embodiments, the biocompatible polymer is a copolymer of polylactic acid and polyglycolic acid, poly(glycerol sebacate) (PGS), poly(ethylenimine), Pluronic (Poloxamers 407, 188), Hyaluron, heparin, agarose, or Pullulan. In some embodiments, the polymer is a copolymer of fumaric/sebacic acid.

The average molecular weight of the polymer used can be from about 20,000 Da to about 500,000 Da.

Any method known in the art can be used to fabricate the nanoparticles for use in the SA-NT of the present invention For example, vaporization methods (e.g., freejet expansion, laser vaporization, spark erosion, electro explosion and chemical vapor deposition), physical methods involving mechanical attrition (e.g., the pearl milling technology developed by Elan Nanosystems), and interfacial deposition following solvent displacement may all be used.

Preferably, the nanoparticles used in the SA-NT of the present invention comprise copolymers of polylactic acid and polyglycolic acid (also known as PLGA). When the nanoparticles used in the SA-NT of the present invention comprise PLGA, the ratio of lactide to glycolide in the PLGA is preferably from about 10:90 to about 90:10, preferably from about 25:75 to about 75:25, and most preferably about 50:50.

When the nanoparticles comprise PLGA, the nanoparticles may be formed as a perfluorobutane polymer microsphere. For example, the nanoparticles used in the SA-NT of the present invention may be HDDS™ (Hydrophobic Drug Delivery System) nanoparticles obtained from Acusphere. These nanoparticles are made by creating an emulsion containing PLGA, a phospholipid and a pore-forming agent. This emulsion is further processed by spray drying to produce small porous microspheres containing gas, having a structure analogous to that of a honeycomb.

The nanoparticles of the present invention may be fabricated, for example, according to the methods described in Examples 1 and 2a.

### Nanoparticle Aggregates

The aggregates used in the SA-NT of the present invention comprise a plurality of constituent nanoparticles. Typically, the aggregates comprise from about 2 to about 10,000 nanoparticles, preferably from about 3 to about 8,000 nanoparticles, preferably from about 7 to about 6,000 nanoparticles, preferably from about 10 to about 6,000 nanoparticles, and preferably from about 20 to about 4,000 nanoparticles.

In the present invention, the aggregates are micro sized aggregates i.e. are of the order of from about 0.1 μm to about 1,000 μm in size. Typically, the aggregates are similar in size to natural platelets. The average diameter of the aggregates is preferably from about 0.5 μm to about 10 μm, preferably from about 1 μm to about 7 μm, preferably from about 1.4 μm to about 4.5 μm, preferably from about 1.8 μm to about 3.5 μm, and preferably from about 2 μm to about 3 μm.

The nanoparticles may bind together into aggregates by one or more types of intermolecular (i.e. non-covalent) force and/or covalent bonds. For example, the nanoparticles may bind together by one or more of electrostatic interactions, dipole-dipole interactions, van der Waal's forces, hydrogen bonds and/or covalent bonds. When the nanoparticles bind together into aggregates by covalent bonds, a cleavable linker may be used. Any cleavable linker known in the art, or as defined herein, may be used.

The strength of binding between nanoparticles may be tuned by increasing or decreasing the degree of interaction between neighbouring nanoparticles. For example, the strength of nanoparticle binding may be increased by modifying the surface of the nanoparticles to include one or more positive and one or more negatively charged groups, thus increasing the degree of electrostatic interaction between neighbouring nanoparticles. Alternatively, the strength of nanoparticle binding may be decreased, for example, by modifying the surface of the nanoparticle to remove electronegative moieties from the surface of the nanoparticles. This would decrease the strength of dipole-dipole and/or hydrogen bonding between neighbouring nanoparticles.

The nanoparticles can be induced to form nanoparticle aggregates by a wide variety of methods available and well known in the art. Many hydrophobic nanoparticles, such PLGA-based nanoparticles, can self-aggregate in aqueous solution (see for example, C. E. Astete et al., J. Biomater. Sci, Polymer Ed. 17:247 (2006)). Alternatively, a concentrated solution of nanoparticles can be spray dried to form aggregates (see for example, Sung, et al., Pharm. Res. 26:1847 (2009) and Tsapis et al., Proc. Natl. Acad. Sci. USA, 99:12001 (2002)). Other methods of forming aggregates include, but are not limited to, the w/o/w emulsion method and the simple solvent displacement method.

The nanoparticle aggregates described herein disaggregate under shear stress conditions. Shear stress is the frictional drag force applied by blood flow, measured in dynes/cm². When blood flows through a vessel, the blood adjacent to the walls of the vessel tends to adhere to the vessel wall, resulting in reduced blood flow and a velocity gradient. The blood velocity in the centre of a vessel is higher than the blood velocity at the edge of the vessel. The differences in blood velocity result in shear stress being applied to cells and particles in the blood. The shear stress increases as the distance to the vessel wall decreases. The shear stress generated by the flowing blood is also applied to molecules or aggregates that are present in the blood. The shear stress experienced by an aggregate is a function of aggregate size—the larger the aggregate, the larger the shear stress that is applied to it.

Under normal physiological conditions shear stress within the brain and peripheral vasculature is tightly regulated between about 15-30 dynes/cm². Following ischaemic stroke, the shear stress in collateral vessels supplying the ischaemic penumbra exceeds this range and may (for example) exceed 100 dynes/cm². Collateral blood flow shear stress was calculated using collateral blood flow velocity and diameter measurements made in Beard, D. J. et al. (2015). “Intracranial Pressure Elevation Reduces Flow through Collateral Vessels and the Penetrating Arterioles they Supply. A Possible Explanation for ‘Collateral Failure’ and Infarct Expansion after Ischemic Stroke”. Journal of Cerebral Blood Flow & Metabolism, 35(5), 861-872, using the equation τ=γ×η, where τ is shear stress, γ is shear rate and η is viscosity. Shear rate was calculated as 8×(blood flow velocity)/(vessel diameter). Published figures for blood viscosity (3 cP) were used.

The aggregates of the SA-NT of the present invention are configured to disaggregate into their component nanoparticles above a predetermined shear stress. Preferably, the aggregates are configured to disaggregate when they flow through areas of elevated shear stress in blood vessels i.e. where the shear stress in the vessels exceeds normal physiological levels. Preferably, aggregates of the SA-NT of the present invention are configured to disaggregate into their constituent nanoparticles in the collateral vessels supplying the penumbra following ischaemic stroke.

Preferably, the aggregates are configured to disaggregate at a predetermined shear stress of greater than about 30 dynes/cm², preferably greater than about 50 dynes/cm², preferably greater than about 75 dynes/cm², and most preferably greater than about 100 dynes/cm². Optionally, the aggregates may be configured to disaggregate at a shear stress of greater than about 125 dynes/cm², and optionally greater than about 150 dynes/cm². In general, disaggregation of the aggregates into constituent nanoparticles occurs when the shear stress experienced by the aggregate is sufficient to overcome the intermolecular forces (such as electrostatic interactions, dipole-dipole interactions, van der Waal's forces and/or hydrogen bonds) binding the nanoparticles together.

The disaggregation of the aggregates may be partial or complete. When the disaggregation is partial, the aggregate may disaggregate by at least 5%, at least 10%, at least 15%, at least 20%, at least 25%, at least 30%, at least 40%, at least 50%, at least 60%, at least 70%, at least 75%, at least 80%, at least 85%, at least 90%, or at least 95% when exposed to the predetermined elevated shear stress. Alternatively, the aggregate may undergo complete disaggregation (i.e. it may fully disaggregate into its constituent nanoparticles) when exposed to the predetermined elevated shear stress.

Suitable nanoparticles for use in the present invention, and aggregates of those nanoparticles, are described in Korin et al. (Science. 2012 Aug. 10; 337(6095):738-42) and WO 2013/185032, the content of which is incorporated herein by reference.

### Vasodilating Agents

The SA-NT according to the present invention is used to selectively deliver vasodilating agents/drugs to the collateral vessels supplying the ischaemic penumbra. Vasodilating agents/drugs are compounds which cause dilation of blood vessels. As used herein, the terms vasodilating agent and vasodilating drug are used interchangeably.

Any vasodilating agent capable of providing dilation of the collateral vessels may be used. Preferably, the one or more vasodilating agents is selected from the group consisting of a nitrate, an angiotensin II receptor blocker (ARB, also referred to as a sartan), a calcium channel blocker (CCB), a selective alpha blocker, a beta1 agonist, a beta2 agonist, a beta-agonist, an ET1 Receptor Antagonist, a Phosphodiesterase 5 inhibitor, or an agonist of small (SK) and intermediate (IK) calcium-activated potassium channels. More preferably, the one or more vasodilating agents is selected from the group consisting of a nitrate, an angiotensin II receptor blocker (ARB, also referred to as a sartan), a calcium channel blocker (CCB), a selective alpha blocker, or a beta1 agonist. Most preferably, the one or more vasodilating agents is selected from the group consisting of a nitrate, an angiotensin II receptor blocker (ARB, also referred to as a sartan), or a calcium channel blocker (CCB).

When the one or more vasodilating agents is a nitrate, the nitrate is preferably nitroglycerin (NG), Nicorandil, Molsidomine/3-Morpholinosydnonimine hydrochloride, Sodium Nitropruside, Isosorbide Mono-nitrate, or Isosorbide di-nitrate, more preferably nitroglycerin (NG), Nicorandil, Molsidomine/3-Morpholinosydnonimine hydrochloride, or Sodium Nitropruside and most preferably nitroglycerin (NG).

When the one or more vasodilating agents is an angiotensin II receptor blocker (ARB, also referred to as a sartan), the ARB is preferably Candesartan, Valsartan, Iosartan, Eprosartan, Olmesartan, Telmisartan, or Irbesartan, more preferably Candesartan, Valsartan, Iosartan, Eprosartan, or Olmesartan, and most preferably Candesartan, or Val sartan.

When the one or more vasodilating agents is a calcium channel blocker (CCB), the CCB is preferably Nimodipine, Lercanidipine, Nifedipine, Felodipine, Nicardipine, Verapamil, Diltiazem, Amlodipine, or Clevidipine, more preferably Nimodipine, Lercanidipine, Nifedipine, Felodipine, Nicardipine, or Verapamil, more preferably Nimodipine, Lercanidipine, Nifedipine, or Felodipine, and most preferably nimodipine.

When the one or more vasodilating agents is a selective alpha blocker, the selective alpha blocker is preferably Doxazosin (Mesilate), Prazosin, or Terazosin, and most preferably Doxazosin (Mesilate).

When the one or more vasodilating agents is a beta1 agonist, the beta1 agonist is preferably Dobutamine, or Dopamine.

When the one or more vasodilating agents is a beta2 agonist, the beta2 agonist is preferably Eformoterol, Indacaterol, Salbutamol, Salmeterol, or Turbutaline.

When the one or more vasodilating agents is a beta-agonist, the beta-agonist is preferably Isoprenaline.

When the one or more vasodilating agents is a ET1 receptor antagonist, the ET1 receptor antagonist is preferably Ambrisentan, Bosentan, or BQ123 and is preferably BQ123.

When the one or more vasodilating agents is a phosphodiesterase 5 inhibitor, the phosphodiesterase 5 inhibitor is preferably Sildenafil, Tadalafil, or Papavarine.

When the one or more vasodilating agents is an agonist of small (SK) and intermediate (IK) calcium-activated potassium channels, the agonist of small (SK) and intermediate (IK) calcium-activated potassium channels is preferably NS309, EBIO, SKA-31, SKA-121, or SKA-111, most preferably NS309.

When the one or more vasodilating agents is not one of the classes listed above, the one or more vasodilating agents is preferably Diazoxide, Hydralazine, Methyldopa, or Minoxidil.

When a plurality of vasodilating agents are used in the SA-NT of the present invention, the vasodilating agents may be from the same class of vasodilating agents, or a plurality of different classes of vasodilating agents (including classes not discussed herein).

Most preferably, the one or more vasodilating agents is nitroglycerin, or nimodipine, or a combination thereof. In one embodiment, the one or more vasodilating agents is nitroglycerin. In another embodiment, the one or more vasodilating agents is nimodipine.

A vasodilating agent may be used alone, or a combination of two or more different vasodilating agents may be used. Where two or more vasodilating agents are used, these may be provided on the same nanoparticle, or on separate nanoparticles which are combined together as a single aggregate. Further, where two or more vasodilating agents are used, these may be provided in the same aggregate, or in separate aggregates.

A vasodilating agent may be provided in the form of a pharmaceutically acceptable salt. As used herein and unless stated otherwise, any reference to a vasodilating agent or a vasodilating drug includes reference to the pharmaceutically acceptable salts.

The one or more vasodilating agents may be coupled to the nanoparticles or the nanoparticle aggregates either before or after aggregation of the nanoparticles into the aggregate of the SA-NT. The one or more vasodilating agents may be coupled to the nanoparticles or nanoparticle aggregates in any method known in the art.

As used herein, with respect to vasodilating agents and nanoparticles or nanoparticle aggregates, the phrase “coupled to” means that the vasodilating agent is entangled, embedded, incorporated, encapsulated, bound to the surface, or otherwise associated with the aggregate or a nanoparticle constituent of the aggregate.

In some embodiments, the one or more vasodilating agents are encapsulated within the aggregate or a nanoparticle constituent of the aggregate. In some embodiments, the one or more vasodilating agents are absorbed or adsorbed on the surface of the aggregate. Thus, one or more vasodilating agents can be associated with the outer surface of the aggregate. This can occur, for example, when the nanoparticles on the outer surface of the aggregate are coupled to the one or more vasodilating agents after forming a nanoparticle aggregate.

In some embodiments, the one or more vasodilating agents are absorbed or adsorbed on the surface of at least a plurality of the nanoparticle constituents of the aggregate. This can occur, for example, when the nanoparticles are coupled to the one or more vasodilating agents before forming a nanoparticle aggregate.

In some embodiments, the one or more vasodilating agents are covalently coupled to the aggregate or nanoparticle constituent of the aggregate. Covalent coupling between molecules of the one or more vasodilating agents and the aggregate or a nanoparticle constituent of the aggregate may be mediated by a linker. Without limitation, any conjugation chemistry known in the art for conjugating two molecules or different parts of a molecule together can be used for linking a vasodilating molecule to a nanoparticle or nanoparticle aggregate. Exemplary linker and/or functional groups for conjugating a vasodilating agent to a nanoparticle or aggregate include, but are not limited to, a polyethylene glycol (PEG, NH₂-PEG_{X}-COOH, which can have a PEG spacer arm of various lengths X, where 1<X<100, e.g. PEG-2K, PEG-5K, PEG-10K, PEG-12K, PEG-15K, PEG-20K, PEG-40K, and the like), maleimide linker, PASylation, HESylation, bis(sulfosuccinimidyl) suberate linker, DNA linker, peptide linker, silane linker, polysaccharide linker, hydrolyzable linker etc. PEG is a preferred linker. Conjugation of a SA-NT aggregate to a therapeutic agent via a PEG linker can be achieved, for example, as described in WO 2013/185032, the content of which is incorporated herein by reference.

In some embodiments, the linker comprises at least one cleavable linking group, i.e. the linker is a cleavable linker as defined herein.

In some embodiments, the one or more vasodilating agents are non-covalently coupled to the aggregate or a nanoparticle constituent of the aggregate. Non-covalent coupling between molecules of the one or more vasodilating agents and the aggregate or to a nanoparticle constituent of the aggregate can be based on ionic interactions, van der Waals interactions, dipole-dipole interactions, hydrogen bonds, electrostatic interactions, and/or shape recognition interactions.

The one or more vasodilating agents do not need to be coupled to nanoparticles before formation of the nanoparticle aggregates. For example, pre-formed nanoparticles can be aggregated in the presence of one or more vasodilating agents. Without wishing to be bound by a theory, the one or more vasodilating agents may then be present in the spaces (or cavities) in the aggregate i.e. the one or more vasodilating agents may be encapsulated within the aggregate.

In the SA-NT of the present invention the nanoparticle aggregates may comprise the one or more vasodilating agents in a broad weight range. For example, the nanoparticle aggregates may comprise from about 0.1% to about 20% by weight of the one or more vasodilating agents. Preferably, the aggregates comprise from about 0.2% to about 10%, preferably from about 0.3% to about 5%, preferably from about 0.4% to about 2.5%, preferably from about 0.8% to about 1.6% about preferably from about 1% to about 1.4% by weight of the one or more vasodilating agents.

When it is intended to use nitroglycerin (or a pharmaceutically acceptable salt thereof) as the one or more vasodilating agents, the nanoparticle aggregates of the SA-NT preferably comprise from about 0.4% to about 2.5%, preferably from about 0.8% to about 1.6%, preferably from about 1% to about 1.4% by weight of the nitroglycerin or the pharmaceutically acceptable salt thereof.

When it is intended to use nitroglycerin as the one or more vasodilating agents, the nanoparticle aggregates comprising nitroglycerin may be fabricated, for example, according to the method described in Example 2a.

The one or more vasodilating agents may be released upon adherence of nanoparticles and/or aggregates to the surface of blood vessels. As set out above, the smaller nanoparticles experience lower drag forces than the larger aggregate particles. This means that the smaller nanoparticles adhere more efficiently than the larger aggregate particles to the surface of the blood vessels through which they are flowing. As a result of the more efficient adherence of smaller nanoparticles, the rate of release of the one or more vasodilating agents to the surface of blood vessels is typically greater for the smaller nanoparticles than for larger aggregate particles.

Thus, the one or more vasodilating agents is typically released at a higher rate and/or in a higher amount when the nanoparticles are disaggregated than when the nanoparticles are aggregated. As such, the SA-NT described herein is able to selectively deliver the one or more vasodilating agents to the surface of blood vessels with elevated shear stress, i.e. the collateral blood vessels supplying the penumbra, following ischaemic stroke.

The Examples described below relate to the use of a SA-NT of the present invention to deliver a vasodilating agent to collateral blood vessels in the brain. FIGS. 1 and 2 (based on Example 3a) show that a large increase in collateral perfusion is observed when nanoparticle aggregates comprising nitroglycerin are administered, as compared to no change in collateral perfusion when nanoparticle aggregates not comprising a vasodilating agent (“blank-NPAs”) are administered.

As set out above, Bath et al. (The Lancet, Volume 393, Issue 10175, pages 1009-1020) recently showed that that transdermal (i.e. systemic) delivery of nitroglycerin, a potent vasodilating agent, did not alter functional outcome in patients suffering ultra-acute stroke. This finding is supported by Example 3b and FIG. 3 herein, which reports that delivering “free” nitroglycerin (i.e. not selectively delivered to the collateral vessels using a SA-NT) to rodents caused only a modest increase in peak collateral perfusion.

In contrast, FIGS. 1-5 each show that delivery of nitroglycerin using a SA-NT according to the present invention causes a significant increase in collateral perfusion. As such, selectively delivering one or more vasodilating agents to the collateral vessels using the SA-NT of the present invention provides an effective approach to improving clinical outcomes in patients suffering acute stroke.

### Administration

Administration of the SA-NT of the present invention may be used to increase blood flow to the brain following a stroke. Typically, the SA-NT increases blood flow to the penumbra following stroke. Typically, the SA-NT dilates collateral vessels, typically this is selective dilation of collateral vessels, such that collateral vessels are dilated to a greater degree than other arteries. Typically, therefore, administration of the SA-NT causes increased perfusion through collateral vessels, which may be a selective increase in perfusion through collateral vessels i.e. with minimal, or no change in mean arterial perfusion. Preferably, the change in mean arterial perfusion following administration of the SA-NT of the present invention is less than about 25% from the pre-administration baseline, preferably less than about 20%, and is preferably less than about 15%.

The SA-NT of the present invention may be administered by any method known in the art. Preferably, the nanoparticles are administered intravenously or intra-arterially, most preferably intravenously.

When the SA-NT of the present invention is administered intravenously, the administration may be as a continuous infusion. Preferably, the infusion is delivered to the patient until after the occlusion is removed and blood flow to the brain is restored. For example, the infusion may be delivered for a time period of up to about 500 minutes, optionally up to about 400 minutes, optionally up to about 300 minutes, optionally up to about 200 minutes, and optionally up to about 100 minutes.

When the SA-NT of the present invention is administered intravenously, the administration may alternatively be as a bolus i.e. as a single discrete dose administered to the patient over a short period of time. Multiple boluses may be administered to the patient over the course of the treatment i.e. from first administration of a bolus until the onset of reperfusion. One or more bolus doses may be combined with a continuous infusion. In general, vasodilating agents with longer half-lives may be administered by a regime comprising a bolus dose, whereas those with shorter half-lives may be administered by a regime comprising a continuous infusion.

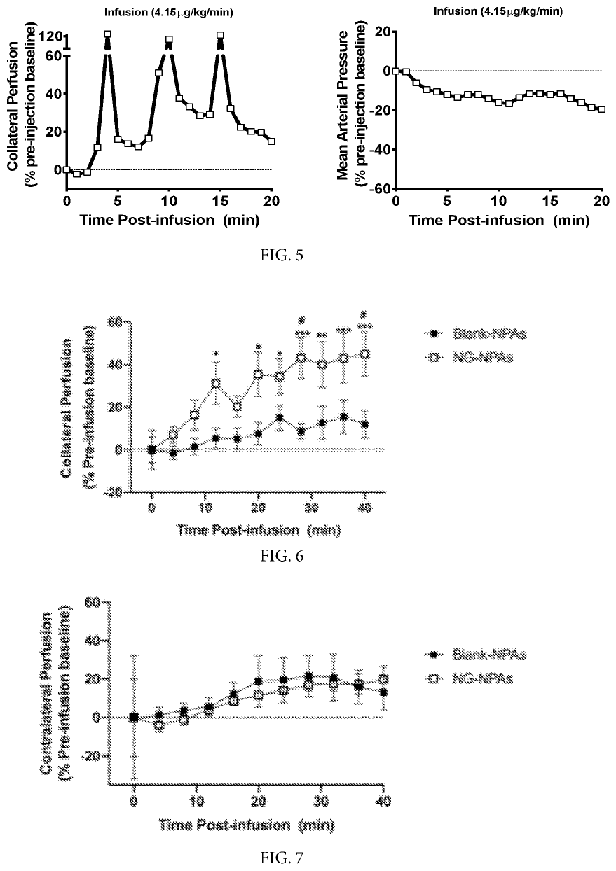

For example, nitroglycerin has a short half-life, whereas nimodipine has a long half-life. As such, when the one or more vasodilating agents comprises nitroglycerin, it is preferable to administer the SA-NT as a continuous infusion (for example, as exemplified in Example 3d and shown in FIGS. 4 and 5). Conversely, when the one or more vasodilating agents comprises nimodipine, it is preferable to administer the SA-NT by a regime comprising a bolus.

The SA-NT of the present invention may be administered at any point following a suspected ischaemic stroke, for example following observance of one or more stroke symptoms selected from sudden numbness or weakness in the face, arm, or leg, sudden confusion, trouble speaking, or difficulty understanding speech, sudden trouble seeing in one or both eyes, sudden trouble walking, dizziness, loss of balance, or lack of coordination, sudden severe headache, complete paralysis of one side of the body, difficulty swallowing (dysphagia) and loss of consciousness.

It is preferable to first administer the SA-NT of the present invention to a patient as soon as possible after onset of stroke symptoms. For example, the SA-NT may be administered as an emergency medication, for example by medical personnel (such as an ambulance paramedic) responding to an emergency call out.

In the case of administration as an emergency medication, the SA-NT of the present invention is preferably administered to a patient within about 4 hours of the onset of stroke symptoms, preferably within about 3 hours of the onset of stroke symptoms, preferably within about 2 hours of the onset of stroke symptoms and more preferably within about 1 hour of the onset of stroke symptoms.

The present invention is particularly useful in treatment of ischaemic stroke. However, the SA-NT may be administered to any patient displaying symptoms of acute stroke, before diagnosis of the type of stroke. Administration can be carried out, for example, without the need for imaging studies to determine the nature of the stroke which the patient has suffered. This is particularly beneficial as it enables administration in an emergency setting. Without wishing to be bound by theory, typical ischaemic stroke treatments may have deleterious effects if administered to a patient suffering a haemorrhagic stroke. However, acute haemorrhagic stroke is not associated with an increase in blood vessel shear stress. As such, SA-NTs according to the present invention do not typically disaggregate or selectively deliver a vasodilating agent to the vessels of a patient suffering a haemorrhagic stroke. As such, the SA-NT of the present invention can be administered in an emergency setting to any patient displaying symptoms of acute stroke, while avoiding the potentially deleterious outcomes to haemorrhagic stroke patients associated with many existing stroke treatments.

In contrast to the present invention, treatments which target the occlusion causing a stroke (e.g. the administration of clot-busting drugs such as t-PA, or thrombectomy) are typically delayed until diagnostic imaging has been carried out. The potential risks involved with such treatments mean that determination of the nature of the stroke is needed prior to treatment. This can delay treatment and brain tissue can be lost during this delay. The ability to treat patients in an emergency setting using the present invention is therefore advantageous compared with treatments targeting the occlusion itself.

### Dosages

It is an aim of the present invention for the SA-NT to result in as large an increase in collateral perfusion as possible, while minimising changes to mean arterial blood pressure. The specific dose of vasodilating agent required to achieve this varies depending on a range of factors including the efficacy of the specific vasodilating agent. It has been shown, for an exemplary vasodilating agent, that delivering the vasodilating agent using a SA-NT of the present invention requires a dose of roughly 1/70^{th} the typical injected dose of ‘free’ vasodilating agent (i.e. not delivered as part of a SA-NT) that is required to increase cerebral blood flow in rats that are not undergoing stroke (as discussed in Hoffman et al. Stroke, Vol 13, No 2, 1982). In particular, FIGS. 4 and 5 show that a substantial increase to cerebral collateral perfusion in rats is obtained by delivering nitroglycerin in a concentration of both 2.75 μg/kg/min and 4.15 μg/kg/min.

Due to the selective delivery of the one or more vasodilating agents to the collateral vessels by a SA-NT of the present invention, it is preferable to administer a dose of between about 0.1% and about 10% of the recommended dose of the ‘free’ vasodilating agent, preferably between about 0.5% and about 8%, preferably between about 1% and about 7%, preferably between about 1.5% and about 6%, preferably between about 2% and about 5%, preferably between about 2.5% and about 4.5%, and most preferably between about 3% and about 4%.

When nitroglycerin is used as the one or more vasodilating agents, it is preferable to administer a dose of from about 0.001 μg/kg/min to about 8 μg/kg/min, preferably from about 0.01 μg/kg/min to about 2 μg/kg/min, preferably from about 0.05 μg/kg/min to about 1 μg/kg/min, preferably from about 0.1 μg/kg/min to about 0.75 μg/kg/min, and most preferably from about 0.15 μg/kg/min to about 0.4 μg/kg/min to a patient.

### Combined Therapies

The SA-NT of the present invention may be administered to a patient in combination with (but typically delivered separately to) a treatment used or intended to remove a blockage in blood flow i.e. a treatment used or intended to initiate reperfusion such as a thrombolysis treatment and/or thrombectomy. This may occur simultaneously, for example when the SA-NT is administered to a patient at the point of reperfusion, or separately; for example when the SA-NT is administered following stroke ischaemia but before reperfusion, and the treatment to initiate reperfusion is started subsequently (either during the administration of the SA-NT of the present invention, or after such administration has stopped). Preferably, the SA-NT is administered to a patient in combination with a thrombolysis treatment and/or thrombectomy.

When the treatment used or intended to initiate reperfusion is used in combination with the SA-NT, the treatment used or intended to initiate reperfusion may be administered at substantially the same time as the SA-NT of the present invention. Alternatively, the treatment used or intended to initiate reperfusion may be administered at a separate time from the SA-NT of the present invention. Typically, the treatment used or intended to initiate reperfusion is administered within about 5 hours of initiating administration of the SA-NT, optionally within about 4 hours, optionally within about 3 hours, optionally within about 2 hours, optionally within about 1 hour, optionally within about 30 minutes and optionally within about 10 minutes of initiating administration of the SA-NT. Typically, the treatment used or intended to initiate reperfusion is administered after about 10 minutes from initiating administration of the SA-NT, optionally after about 30 minutes, optionally after about 1 hour, optionally after about 2 hours, optionally after about 3 hours, optionally after about 4 hours, and optionally after about 5 hours from initiating administration of the SA-NT. Preferably, the thrombolysis treatment is selected from application of blood thinning agents or application of lysis agents to the patient. Suitable blood thinning agents may be selected from Coumadin™ (warfarin); Pradaxa™ (dabigatran); Xareito™ (rivaroxaban) and Eliquis™ (apixaban), Fondaparinux, unfractionated heparin, low molecular weight heparins including but not limited to enoxaparin and deltaparin, thrombolytic agents including but not limited to Streptokinase (SK), Urokinase, Lanoteplase, Reteplase, Staphylokinase, Tenecteplase and Alteplase, or antiplatelet drugs such as aspirin, clopidogreal or ticagrelor.

The present invention may also be used in combination with delivery of one or more neuroprotective drugs. The combination of selectively-applied vasodilator treatment and administration of one or more neuroprotective drugs may enhance the efficacy of the one or more neuroprotective drugs by increasing the rate of delivery of the one or more neuroprotective drugs to the brain via collateral blood vessels.

Preferred classes of neuroprotective drugs that may be used in combination with the SA-NT of the present invention include free radical scavengers, ion channel interaction/excitotoxicity blockade therapies and immune modulation/anti-inflammatory therapies (see, for example, Rajah et al. “Experimental neuroprotection in ischemic stroke: a concise review”. Neurosurg Focus. 2017 April; 42(4)). Preferred neuroprotective drugs that may be used in combination with the SA-NT of the present invention are NXY-059, NA-1, Interleukin-1 receptor antagonist and uric acid. These preferred neuroprotective drugs are known in the art to be well tolerated in clinical stroke patients.

The one or more neuroprotective drugs may be combined with the vasodilating agent in the SA-NT of the present invention, or they may be delivered separately (i.e. not as part of the SA-NT).

When one or more neuroprotective drugs is combined with a vasodilating agent in a SA-NT, the one or more neuroprotective drugs may be provided on the same nanoparticle as the vasodilating agent, or on separate nanoparticles to the vasodilating agent which are combined together as a single aggregate. Similarly, where both neuroprotective drugs and vasodilating agents are used, these may be provided in the same aggregate, or in separate aggregates.

When the one or more neuroprotective drugs is used in combination with the SA-NT but delivered separately, the one or more neuroprotective drugs may be administered at substantially the same time as the SA-NT of the present invention. Alternatively, the one or more neuroprotective drugs may be administered within about 10 minutes of administration of the SA-NT, optionally within about 30 minutes, optionally within about 1 hour, optionally within about 2 hours and optionally within about 3 hours of delivery of the SA-NT of the present invention. Alternatively, the one or more neuroprotective drugs may be administered before administration of the SA-NT of the present invention, for example within about 10 minutes, optionally within about 30 minutes, optionally within about 1 hour, optionally within about 2 hours and optionally within about 3 hours before delivery of the SA-NT.

In the present invention, the SA-NT may be used in combination with both a therapy used or intended to remove a blockage in blood flow (such as thrombolysis and/or thrombectomy) and a neuroprotective therapy such as administration of one or more neuroprotective drugs.

### Compositions

The present invention also relates to compositions for use in treating stroke, wherein the composition comprises a SA-NT as defined herein in combination with one or more pharmaceutically acceptable excipients, carriers or diluents.

Suitable excipients, carriers and diluents can be found in standard pharmaceutical texts. See, for example, Handbook for Pharmaceutical Additives, 3rd Edition (eds. M. Ash and I. Ash), 2007 (Synapse Information Resources, Inc., Endicott, New York, USA) and Remington: The Science and Practice of Pharmacy, 21st Edition (ed. D. B. Troy) 2006 (Lippincott, Williams and Wilkins, Philadelphia, USA).

Excipients for use in the compositions of the invention include, but are not limited to microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia.

Pharmaceutical carriers include sterile aqueous media and various non-toxic organic solvents, and the like. Pharmaceutically acceptable carriers include gums, starches, sugars, cellulosic materials, and mixtures thereof. The preparation can also be administered, for example, by intravenous, intra-arterial, or intramuscular injection of a liquid preparation.

Further, as used herein “pharmaceutically acceptable carriers” are well known to those skilled in the art and include, but are not limited to, 0.01-0.1 M and preferably 0.05 M phosphate buffer or 0.9% saline. Additionally, such pharmaceutically acceptable carriers maybe aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Other examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminium hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutically acceptable parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Pharmaceutically acceptable carriers for controlled or sustained release compositions administrable according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Pharmaceutically acceptable carriers include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski and Davis, Soluble Polymer-Enzyme Adducts, Enzymes as Drugs, Hocenberg and Roberts, eds., Wiley-Interscience, New York, N.Y., (1981), pp 367-383). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

As used herein, the terms “drug”, “drug substance”, “active pharmaceutical ingredient”, and the like, refer to a compound that may be used for treating a patient in need of treatment.

As used herein, the term “excipient” refers to any substance that may influence the bioavailability of a drug, but is otherwise pharmacologically inactive.

As used herein, the term “pharmaceutically acceptable” refers to species which are within the scope of sound medical judgment suitable for use in contact with the tissues of a patient without undue toxicity, irritation, allergic response, and the like, commensurate with a reasonable benefit-to-risk ratio, and effective for their intended use.

As used herein, the term “pharmaceutically acceptable salts” are those salts which can be administered as drugs or components of pharmaceutical compositions to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminium salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared. Additional salts particularly useful for pharmaceutical preparations are described in Berge S. M. et al., “Pharmaceutical salts,” J. Pharm. Sci. 1977 January; 66(1):1-19.

As used herein, the term “pharmaceutical composition” refers to the combination of one or more drug substances and one or more excipients.

As used herein, the term “patient” as used herein refers to a human or non-human mammal. Examples of non-human mammals include livestock animals such as sheep, horses, cows, pigs, goats, rabbits and deer; and companion animals such as cats, dogs, rodents, and horses.

As used herein, the term “body” as used herein refers to the body of a patient as defined above.

As used herein, the term “therapeutically effective amount” of a drug refers to the quantity of the drug or composition that is effective in treating a patient and thus producing the desired therapeutic or ameliorative effect. The therapeutically effective amount may depend on the weight and age of the patient and the route of administration, among other things.

As used herein, the term “treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

As used herein, the term “treatment” refers to the act of “treating”, as defined above.

As used herein, the term “thrombolysis” refers to removal of a blockage to blood flow, for example a blood clot, using chemical means, for example through the use of thrombolytic agents.

As used herein, the term “thrombectomy” refers to removal of a blockage to blood flow, for example a blood clot, using mechanical means.

As used herein, the term “bolus” refers to a single administration of a relatively large discrete dose of a drug given intravenously and rapidly.

As used herein, the term “continuous infusion” refers to continuous intravenous administration of a drug over a set period of time.

As used herein, the term “biocompatible” means exhibiting essentially no cytotoxicity or immunogenicity while in contact with body fluids or tissues.

As used herein, the term “polymer” refers to oligomers, co-oligomers, polymers and co-polymers e.g. random block, multiblock, star, grafted, gradient copolymers and combinations thereof.

As used herein, the term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also non-inflammatory when employed in situ.

As used herein, the term “cleavable linking group” is a chemical group which is stable under one set of conditions, but which is cleaved under a different set of conditions to release the two parts the linker is holding together. Cleavable linking groups are susceptible to cleavage agents, e.g., hydrolysis, pH, elevated shear stress, redox potential, temperature, radiation, sonication, or the presence of degradative molecules (e.g., enzymes or chemical reagents), and the like. Exemplary cleavable linking groups include, but are not limited to, hydrolyzable linkers, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(OR)—S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linking groups (e.g., hydrazones, esters, and esters of amino acids, —C═NN— and —OC(O)—); ester-based cleavable linking groups (e.g. —C(O)O—); peptide-based cleavable linking groups, (e.g., linking groups that are cleaved by enzymes such as peptidases and proteases in cells, e.g. —NHCHRAC(O)NHCHR BC(O)—, where RA and RB are the R groups of the two adjacent amino acids). A peptide based cleavable linking group comprises two or more amino acids. A peptide-based cleavage linkage comprises the amino acid sequence that is the substrate for a peptidase or a protease.

“Nitroglycerin” or “NG” as used herein is also known as and is equivalent to “glyceryl trinitrate” or “GTN”.

As used herein, the term “nitroglycerin nanoparticle aggregate” or “NG-NPA” refers to a SA-NT according to the present invention comprising nitroglycerin as a vasodilating agent.

As used herein the term “comprising” means “consisting at least in part of”. When interpreting each statement in this specification that includes the term “comprising”, features other than that or those prefaced by the term may also be present. Related terms such as “comprise” and “comprises” are to be interpreted in the same manner.

### EXAMPLES

### Example 1—Nanoparticle Aggregate Preparation

Nanoparticles (NPs) were prepared from PLGA (50:50, 17 kDa, acid terminated; Lakeshore Biomaterials, AL) using a simple solvent displacement method. The fluorescent hydrophobic dye, coumarin-6, was included in the NPs to enable visualization and quantitation in this study.

1 mg/ml of polymer was dissolved with 0.1 wt. % coumarin in dimethyl sulfoxide (DMSO, Sigma, MO), dialyzed against water at room temperature, and the nanoparticles were allowed to form by solvent displacement and subsequent self-assembly in aqueous solution.

Fabrication of NP Aggregates: The PLGA NPs were centrifuged and concentrated to a 10 mg/ml suspension in water and 1 mg/ml L-leucine (Spectrum Chemicals & Laboratory Products, CA) was added. NP aggregates were prepared by a spray-drying technique using a Mobile Minor spray dryer (Niro, Inc.; Columbia, MD). The aqueous leucine-NP suspension was infused separately from the organic phase (ethanol) at a ratio of 1.5:1 and mixed in-line immediately prior to atomization. The inlet temperature was 80° C. and the liquid feed rate was 50 ml/min; gas flow rate was set at 25 g/min and nozzle pressure was 40 psi. Spray-dried powders were collected in a container at the outlet of the cyclone. SA-NTs suspensions were formed by reconstituting the powders in water at desired concentrations. Aggregate suspensions were filtered through 20 μm filters to filter out any oversized aggregates; centrifugation (2000 g for 5 min) followed by washing also was used to remove single unbound NPs. Dynamic Light Scattering (DLS) was used to determine the size of the NPs in dilute solutions using a zeta particle size analyzer (Malvern instruments, UK) operating with a HeNe laser, 173° back scattering detector. Samples were prepared at 1 mg/ml concentration in PBS buffer at pH 7.4.

### Example 2a—Nitroglycerin Nanoparticle (NG-NP) Preparation

Nanoparticles (NPs) were prepared from PLGA (50:50, acid terminated; GMP grade PLGA Poly(D,L-lactide-co-glycolide) from Durect Lactel) using a single emulsion solvent evaporation method. 1% (w/v) polyvinyl alcohol (PVA, Sigma-Aldrich) solution sterilized by filtration was prepared in milli-Q water and used as the aqueous phase. 50 mg/mL PLGA polymer dissolved in dichloromethane (Sigma-Aldrich) was used as the organic phase. 5 mg/mL Nitroglycerin (American Regent-Nitroglycerin Injection, USP grade) solution transferred into the organic phase. The organic phase containing PLGA and nitroglycerin solution was mixed in a beaker containing 50 mL PVA containing aqueous phase. This solution mixture was sonicated by using a probe sonicator (Q-Sonica modelQ700) for 1.5 min at 40 AMP intensity inside a 4° C. cold room. The stock solution was dialyzed overnight against Milli-Q water using 100 KD CE tubing (Spectrum-labs). The sample concentration was determined by performing dry weight analysis (triplicates of 0.5 mL of the suspension dried in 120° C. oven and average was used to calculate the total nanoparticle yield, >90% by weight). The size distribution of the formed NPs were characterized using Dynamic Light Scattering (DLS). The Z-average mean size of the NPs was found to be 185.6 nm, with a standard deviation of 75.18. The mode average size of the NPs was found to be 210.8 nm

### Example 2b—Nitroglycerin Nanoparticle Aggregate (NG-NPA) Preparation

The NG-NP suspension was diluted into 5 mg/mL by addition of milli-Q water. 2 mg/mL L-leucine (Spectrum Chemicals & Laboratory Products, CA) used as the aqueous phase. NG nanoparticle aggregates prepared by a spray-drying technique using a benchtop Buchi 290 spray dryer (Buchi-290, Switzerland) parts were sterilized by autoclaving. The aqueous leucine-NP suspension was infused separately from the organic phase (ethanol): NG-NP suspension mixture at a ratio of 1.5:1 and mixed in-line immediately prior to atomization. The inlet temperature was 110° C. and the liquid feed rate was 6 mL/min; aspirated with 60 mbar pressure. Spray dried powders were collected in a container at the outlet of the cyclone and stored in desicator at −20° C. The nitroglycerin loading inside nanoparticle aggregates was 1.2% by weight, measured by high-pressure liquid chromatography (HPLC). For size measurement, samples were prepared at 1 mg/mL concentration in PBS buffer at pH 7.4 and measured with Malvern laser diffraction instrument. The median size of the nanoparticle aggregates was found to be 2.5 μm.

### Example 3—Use of Nanoparticles Comprising Nitroglycerin to Increase Collateral Perfusion in Rodents

Animal experiments were performed on male Spontaneously Hypertensive Rats (SHRs) weighing 300-350 g (Harlan, Bicester, UK). All animals were housed in a 12 h light/dark cycle and had ad libitum access to food and water prior to experiments. All procedures conformed to the Animal (Scientific Procedures) Act 1986 (UK) and the National Institutes of Health guidelines for care and use of laboratory animals and were approved by the University of Oxford Animal Ethics Committee, the Home Office (UK).

### Anaesthesia and Monitoring

Rats were anesthetized with 5% isoflurane in O₂/N₂ (1:3) and maintained with 1% to 2% isoflurane. Core temperature was maintained at 37° C. by a thermocouple rectal probe and warming plate (Harvard Apparatus, UK). Incision sites were shaved, cleaned, and injected subcutaneously with 2 mg/kg 0.05% Bupivacaine (Aspen, UK). A femoral arterial line was used for continuous arterial pressure monitoring.

### Experimental Stroke Model

Rats underwent middle cerebral artery occlusion (MCAo/stroke) using the silicone-tipped intraluminal thread occlusion method (as set out in Spratt et al. (2006): “Modification of the method of thread manufacture improves stroke induction rate and reduces mortality after thread-occlusion of the middle cerebral artery in young or aged rats.” J Neurosci Methods), using 4-0 monofilament occluding threads with 4 mm length x 0.35 mm diameter silicone tips. The filament was advanced into the right external carotid artery stump and up the internal carotid artery to occlude the origin of the right MCA.

### Measurements of Changes in Core and Collateral Blood Flow Using Multi-Site Dual Laser Doppler Probes

Laser Doppler flowmetry (LDF) (Oxford Optronix, Oxford, UK) was used to measure changes in cerebral blood flow (CBF) in both the MCA and collateral arterial territories using dual probes. The animal's head was secured with ear bars in a stereotaxic frame. Probe 1 was placed+4 mm lateral of midline and −2 mm posterior of Bregma to measure changes in core MCA CBF. Probe 2 was placed+3 mm lateral of midline and +2 mm anterior of Bregma to measure changes in CBF supplied by collateral vessels within the border zone between the anterior cerebral artery (ACA) and MCA perfusion territories. Middle cerebral artery occlusion was confirmed by >70% decrease in LDF signal from baseline in Probe 1.

### Drug Administration

### Example 3a

Prior to MCAo, the femoral vein was cannulated with 2-French silicon tubing. Animals were randomized to receive intravenous bolus and then infusion of blank nanoparticle aggregates (NPA control, 1 mg in Saline n=6) or bolus and then infusion of nitroglycerin nanoparticle aggregates (NG-NPAs, 12.5 μg nitroglycerin in 1 mg NPA in saline, n=6) I.V, 30 minutes after MCAo. Collateral perfusion was measured as a change from pre-injection baseline. Changes in collateral perfusion are shown in FIG. 1. A comparison of the average collateral perfusion of the blank NPAs and the NG-NPAs is shown in FIG. 2.

Blank NPAs did not significantly change collateral perfusion. NG-NPA administration significantly increased collateral blood perfusion by an average of 40% above baseline at 4 minutes after onset of administration (FIG. 1). NG-NPAs administration also increased the average collateral perfusion over the treatment period (FIG. 2).

### Example 3b

Prior to MCAo, the femoral vein was cannulated with 2-French silicon tubing. One spontaneously hypertensive rat (SHR) received intravenous bolus over 1 minute of increasing doses of free nitroglycerin (NG, 12.5, 25, 50 and 100 m) that had not been packaged in NPAs, commencing 30 minutes after MCAo. There was a washout period of 5 half-lives of NG between administration of each dose. Collateral perfusion was measured as a % change from pre-injection baseline. Results are shown in FIG. 3. Peak collateral blood flow increase was 2.5 fold higher in the NG-NPA group compared to the equivalent dose of nitroglycerin not packaged into NPA. Furthermore, increasing doses of “free” nitroglycerin showed no additional increase in collateral perfusion (FIG. 3).

### Example 3c

Prior to MCAo, the femoral artery and the femoral vein was cannulated with 2-French silicon tubing. Mean arterial pressure was measured continuously through the femoral artery catheter. Collateral perfusion was measured as a % change from pre-injection baseline. One SHR received intravenous infusion of NG-NPAs (containing 25 μs nitroglycerin in 2 mg NPA in saline at 2 ml/hour=NG dose of 2.75 μg/kg/min), commencing 30 minutes after MCAo. Changes in collateral perfusion and mean arterial pressure for this SHR are shown in FIG. 4.

Another SHR received an intravenous infusion of NG-NPAs (containing 25 μg nitroglycerin in 2 mg NPA in saline at 3 ml/hour=NG dose of 4.15 μg/kg/min), commencing 30 minutes after MCAo. Changes in collateral perfusion and mean arterial pressure for this SHR are shown in FIG. 5.

### Example 4—Use of Nanoparticles Comprising Nitroglycerin to Increase Collateral Perfusion in Rodents

Animal experiments were performed on male Spontaneously Hypertensive Rats (SHRs) weighing 280-310 g (ARC, Perth, Australia). All animals were housed in a 12 h light/dark cycle and had ad libitum access to food and water prior to experiments. Experiments were approved by the Animal Care and Ethics Committee of the University of Newcastle (Protocol #A-2020-003) and were in accordance with the requirements of the Australian Code of Practice for the Care and Use of Animals for Scientific Purposes.

### Anaesthesia and Monitoring

Rats were anesthetized with 5% isoflurane in O₂/N₂ (1:3) and maintained with 1% to 2% isoflurane. Core temperature was maintained at 37° C. by a thermocouple rectal probe and warming plate. Incision sites were shaved, cleaned, and injected subcutaneously with 2 mg/kg 0.05% Bupivacaine. A femoral arterial line was used for continuous arterial pressure monitoring.

### Experimental Stroke Model

Rats underwent middle cerebral artery occlusion (MCAo/stroke) using the silicone-tipped intraluminal thread occlusion method (as set out in Spratt et al. (2006): “Modification of the method of thread manufacture improves stroke induction rate and reduces mortality after thread-occlusion of the middle cerebral artery in young or aged rats.” J Neurosci Methods), using 4-0 monofilament occluding threads with 4 mm length×0.35 mm diameter silicone tips. The filament was advanced into the right external carotid artery stump and up the internal carotid artery to occlude the origin of the right MCA.

### Measurements of Changes in Collateral Blood Flow in Stroke (Right) Hemisphere and Cerebral Blood in the Control (Left) Hemisphere Using Laser Speckle Contrast Imaging

Laser speckle contrast imaging (RWD Life Sciences) through a bilateral thinned-skull cranial windows was used to measure changes in cerebral blood flow (CBF) in collateral arterial territory on the right (stroke) side of the brain and in the equivalent region on the left (control/contralateral) side of the brain. The animal's head was secured with ear bars in a stereotaxic frame. Cranial windows (4 mm×4 mm) were created bilaterally starting at 1 mm posterior of bregma and 1 mm lateral to midline. Regions of interest on laser speckle imaging software were placed 2 mm behind bregma and 3 mm lateral from midline on the right-hand side to measure changes in CBF supplied by collateral vessels within the border zone between the anterior cerebral artery (ACA) and MCA perfusion territories. Another region of interest was placed in the equivalent region (i.e., 2 mm behind bregma and 3 mm lateral from midline) on the left hand (control/contralateral) hemisphere.

### Drug Administration

Prior to MCAo, the femoral artery and the femoral vein were cannulated. Mean arterial pressure was measured continuously through the femoral artery catheter. Collateral and control hemisphere perfusion were measured as a % change from pre-injection baseline.

Animals were randomized to receive intravenous infusion of blank nanoparticle aggregates (NPA control, 4 mg in 2 ml of Saline, n=7) or infusion of nitroglycerin nanoparticle aggregates (NG-NPAs, 50 μg nitroglycerin in 4 mg NPA in 2 ml of saline at 2.8-3 ml/hr=4 μg/kg/min of nitroglycerin, n=7), commencing 25 minutes after MCAo. Infusion continued for 45 minutes until reperfusion was induced by retracting the occluding thread. Animals were recovered for 24 hours post-surgery at which time they were euthanised and their brain was analysed for final stroke (infarct) size.

### Results

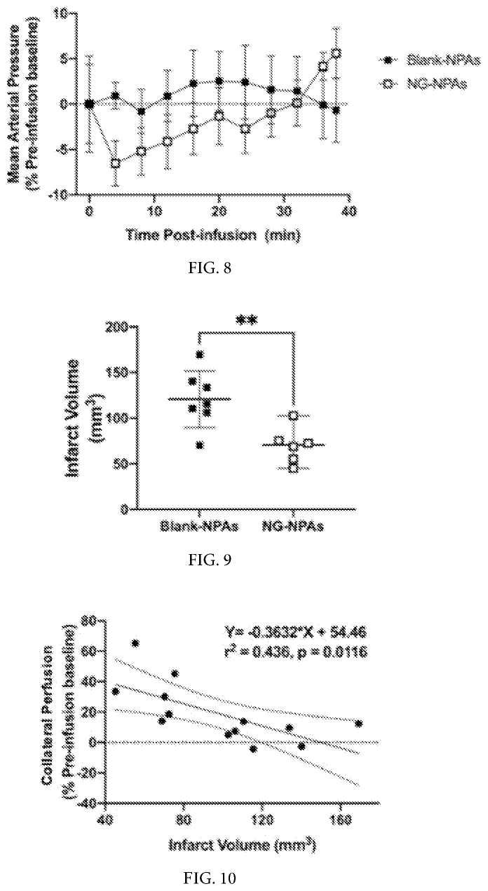

NG-NPA administration significantly increased collateral blood perfusion and blank NPAs did not significantly change collateral perfusion (FIG. 6). NG-NPAs had no effect on perfusion in the corresponding region of the control/contralateral hemisphere (FIG. 7). NG-NPAs did not significantly drop blood pressure (FIG. 8). NG-NPA treatment significantly reduced the size of the stroke at 24 hours (FIG. 9). There is a significantly inverse correlation between the degree of change in collateral perfusion and infarct volume when both groups are combined (FIG. 10). This inverse relationship between collateral perfusion and infarct volume confirms the protective effect of improved collateral perfusion on stroke outcome. These results confirm that NG-NPAs enhance collateral perfusion, are being targeted specifically to the collaterals (no change in contralateral/control hemisphere perfusion and blood pressure) and improve stroke outcome.

### Example 5—Effect of Free Nitroglycerin on Perfusion and Blood Pressure in Rodents

Animal experiments were performed on male Spontaneously Hypertensive Rats (SHRs) weighing 280-310 g (ARC, Perth, Australia). All animals were housed in a 12 h light/dark cycle and had ad libitum access to food and water prior to experiments. Experiments were approved by the Animal Care and Ethics Committee of the University of Newcastle (Protocol #A-2020-003) and were in accordance with the requirements of the Australian Code of Practice for the Care and Use of Animals for Scientific Purposes.

### Anaesthesia and Monitoring

Rats were anesthetized with 5% isoflurane in O₂/N₂ (1:3) and maintained with 1% 25 to 2% isoflurane. Core temperature was maintained at 37° C. by a thermocouple rectal probe and warming plate. Incision sites were shaved, cleaned, and injected subcutaneously with 2 mg/kg 0.05% Bupivacaine. A femoral arterial line was used for continuous arterial pressure monitoring.

### Experimental Stroke Model

Rats underwent middle cerebral artery occlusion (MCAo/stroke) using the silicone-tipped intraluminal thread occlusion method (as set out in Spratt et al. (2006): “Modification of the method of thread manufacture improves stroke induction rate and reduces mortality after thread-occlusion of the middle cerebral artery in young or aged rats.” J Neurosci Methods), using 4-0 monofilament occluding threads with 4 mm length×0.35 mm diameter silicone tips. The filament was advanced into the right external carotid artery stump and up the internal carotid artery to occlude the origin of the right MCA.

### Measurements of Changes in Collateral Blood Flow in Stroke (Right) Hemisphere and Cerebral Blood in the Control (Left) Hemisphere Using Laser Speckle Contrast Imaging

Laser speckle contrast imaging (RWD Life Sciences) through a bilateral thinned-skull cranial windows was used to measure changes in cerebral blood flow (CBF) in collateral arterial territory on the right (stroke) side of the brain and in the equivalent region on the left (control/contralateral) side of the brain. The animal's head was secured with ear bars in a stereotaxic frame. Cranial windows (4 mm×4 mm) were created bilaterally starting at 1 mm posterior of Bregma and 1 mm lateral to midline. Regions of interest on laser speckle imaging software were placed 2 mm behind bregma and 3 mm lateral from midline on the right-hand side to measure changes in CBF supplied by collateral vessels within the border zone between the anterior cerebral artery (ACA) and MCA perfusion territories. Another region of interest was placed in equivalent region of interest was placed on the left hand (control/contralateral) hemisphere.

### Drug Administration

Prior to MCAo, the femoral artery and the femoral vein were cannulated. Mean arterial pressure was measured continuously through the femoral artery catheter. Collateral and control hemisphere perfusion were measured as a % change from pre-injection baseline.

4 μg/Kg/Min Nitroglycerin (GTN)

Animals were randomized to receive intravenous infusion of saline (control, 0.3 ml/hr n=6) or infusion of free nitroglycerin (free-GTN, 0.25 μg/μl in saline, at 300 μl/h=4 μg/kg/min, n=6), commencing 25 minutes after MCAo. Infusion continued for 45 minutes until reperfusion was induced by retracting the occluding thread.

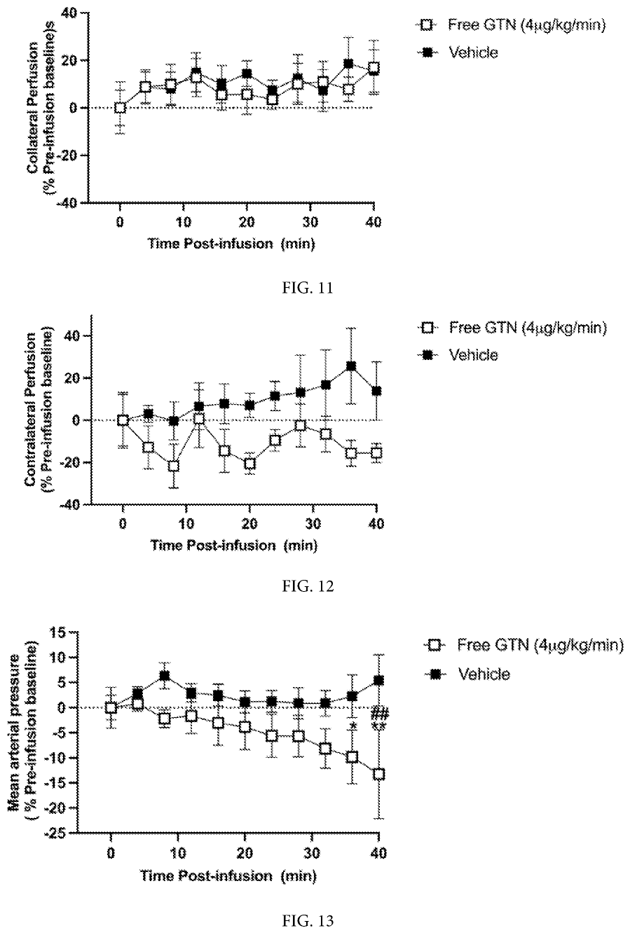

4 μg/kg/min free-GTN administration did not change collateral blood perfusion (FIG. 11). 4 μg/kg/min free-GTN had no significant effect on perfusion in the corresponding region of the control/contralateral hemisphere (FIG. 12). 4 μg/kg/min free-GTN dropped blood pressure (FIG. 13).

These results confirm that GTN administered as free drug, has no effect on collateral perfusion but causes hypotension.

40 μg/Kg/Min Nitroglycerin (GTN)

Animals received intravenous infusion of free nitroglycerin (free-GTN, 2.5 μg/μl in saline, at 300 μl/h=40 μg/kg/min, n=4), commencing 25 minutes after MCAo. Infusion continued for 45 minutes until reperfusion was induced by retracting the occluding thread. Vehicle control (saline) was the same control group used for 4 μg/kg/min studies (n=6).

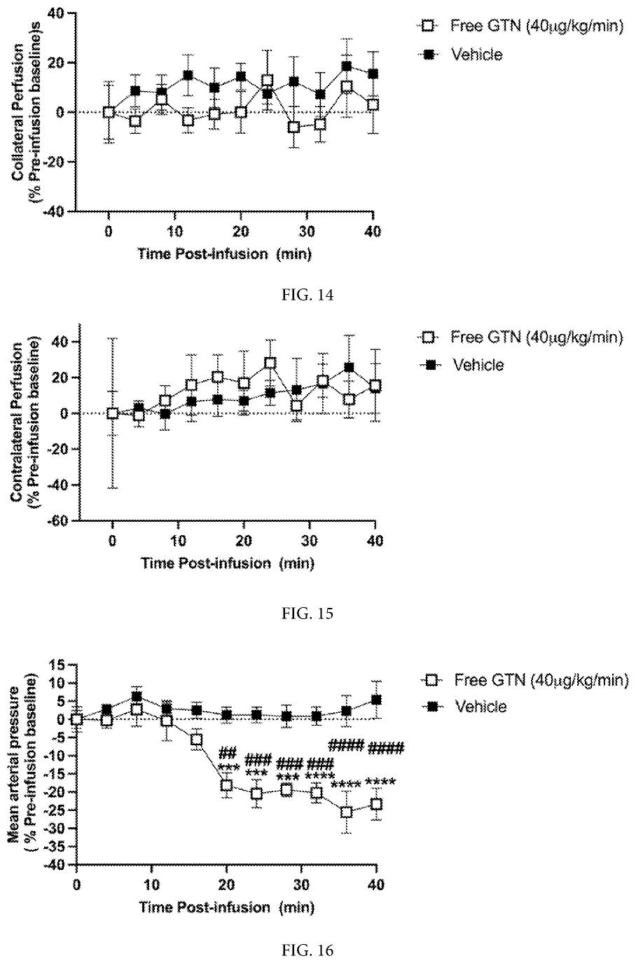

40 μg/kg/min free-GTN administration did not change collateral blood perfusion (FIG. 14). 40 μg/kg/min free-GTN had no significant effect on perfusion in the corresponding region of the control/contralateral hemisphere (FIG. 15). 40 μg/kg/min free-GTN significantly dropped blood pressure (FIG. 16).

These results confirm that GTN administered as free drug at doses equivalent to 10 times higher than the dose in NG-NPAs has no effect on collateral perfusion but causes a dose-dependent hypotension. These results highlight that simply increasing the dose of free GTN from 4 μg/kg/min to 40 μg/kg/min is unable to enhance collateral perfusion, most likely due to the hypotension resulting from free GTN dilating systemic blood vessels. It is therefore not possible to determine the equivalent dose of free drug needed to cause the same collateral enhancing effects of 4 μg/kg/min GTN that is packaged into NG-NPAs, and thus it is not possible to determine the fold enhancement of efficacy NG-NPAs give for collateral enhancement.

Taken together with the results of Example 4, these results demonstrate that NG-NPAs can take GTN from being an ineffective collateral enhancing therapy to a highly effective collateral enhancing therapy due to its selective delivery to collateral vessels and lack of systemic hypotension.

While specific embodiments of the invention have been described above, it will be appreciated that the invention may be practiced otherwise than as described. The descriptions above are intended to be illustrative, not limiting. Thus it will be apparent to one skilled in the art that modifications may be made to the invention as described without departing from the scope of the claims set out below.

1. A shear-activated nanotherapeutic (SA-NT) for use in treating stroke by increasing blood supply to the brain via collateral vessels, wherein the SA-NT comprises an aggregate comprising a plurality of nanoparticles, the aggregate further comprising one or more vasodilating agents or pharmaceutically acceptable salts thereof;
wherein the aggregate is configured to disaggregate above a predetermined shear stress.

2. The shear-activated nanotherapeutic for use according to claim 1, wherein the one or more vasodilating agents is selected from the group consisting of a nitrate, an angiotensin II receptor blocker, a calcium channel blocker, a selective alpha blocker, a beta1 agonist, a beta2 agonist, a beta-agonist, an ET1 Receptor Antagonist, a Phosphodiesterase 5 inhibitor, or a agonist of small (SK) and intermediate (IK) calcium-activated potassium channels.

3. The shear-activated nanotherapeutic for use according to claim 1 or claim 2, wherein the one or more vasodilating agents is selected from the group consisting of a nitrate, an angiotensin II receptor blocker, or a calcium channel blocker.

4. The shear-activated nanotherapeutic for use according to any one of claims 1 to 3, wherein the one or more vasodilating agents comprises nitroglycerin.

5. The shear-activated nanotherapeutic for use according to any one of claims 1 to 4, wherein the one or more vasodilating agents comprises nimodipine.

6. The shear-activated nanotherapeutic for use according to any one of claims 1 to 5, wherein the SA-NT is configured to release its constituent nanoparticles at a shear stress of greater than about 100 dynes/cm².

7. The shear-activated nanotherapeutic for use according to any one of claims 1 to 6, wherein the nanoparticles comprise copolymers of polylactic acid and polyglycolic acid.

8. The shear-activated nanotherapeutic for use according to any one of claims 1 to 7, wherein the one or more vasodilating agents or pharmaceutically acceptable salts thereof is encapsulated in the nanoparticle, is adsorbed on the surface of the nanoparticles, or is covalently linked to the nanoparticle.

9. The shear-activated nanotherapeutic for use according to any one of claims 1 to 8, wherein the one or more vasodilating agents or pharmaceutically acceptable salts thereof is released at a higher rate and/or in a higher amount when the nanoparticles are disaggregated than when the nanoparticles are aggregated.

10. A composition for use in treating stroke by increasing blood supply to the brain via collateral vessels, wherein the composition comprises a shear-activated nanotherapeutic (SA-NT) as defined in any one of claims 1 to 9 in combination with one or more pharmaceutically acceptable excipients, carriers and/or diluents.

11. The shear-activated nanotherapeutic for use according to any one of claims 1 to 9 or the composition for use according to claim 10, wherein the SA-NT or the composition is administered intravenously.

12. The shear-activated nanotherapeutic or the composition for use according to any one of claims 1 to 11, wherein the SA-NT or the composition is administered as a continuous infusion.

13. The shear-activated nanotherapeutic or the composition for use according to any one of claims 1 to 11, wherein the SA-NT or the composition is administered as a bolus.

14. The shear-activated nanotherapeutic or the composition for use according to any one of claims 1 to 13, wherein the stroke is ischaemic stroke.

15. The shear-activated nanotherapeutic or the composition for use according to any one of claims 1 to 14, wherein the treatment increases blood flow to the penumbra.

16. The shear-activated nanotherapeutic or the composition for use according to any one of claims 1 to 15, wherein the SA-NT or the composition is administered in combination with a neuroprotective therapy.

17. The shear-activated nanotherapeutic or the composition for use according to any one of claims 1 to 16, wherein the SA-NT or the composition is administered in combination with one or more additional therapies selected from thrombolysis therapies and thrombolytic therapies; wherein the additional therapy is not administered as part of the SA-NT.

18. A method of treating stroke by increasing blood supply to the brain via collateral vessels comprising administering a shear-activated nanotherapeutic (SA-NT) as defined in any one of claims 1 to 9, or a composition as defined in claim 10 to a patient in need thereof.

19. Use of a shear-activated nanotherapeutic (SA-NT) as defined in any one of claims 1 to 9, or a composition as defined in claim 10 for the manufacture of a medicament for treating stroke by increasing blood supply to the brain via collateral vessels.

20. A shear-activated nanotherapeutic (SA-NT) comprising an aggregate comprising a plurality of nanoparticles; wherein the aggregate is configured to disaggregate above a predetermined shear stress; and wherein the aggregate further comprises from about 0.4% to about 2.5% by weight of nitroglycerin, or a pharmaceutically acceptable salt thereof.

21. The shear-activated nanotherapeutic according to claim 20, wherein the aggregate comprises from about 0.8% to about 1.6% by weight of nitroglycerin, or a pharmaceutically acceptable salt thereof.

22. The shear-activated nanotherapeutic according to claim 20 or 21, wherein the aggregate comprises about 1% to about 1.4% by weight of nitroglycerin, or a pharmaceutically acceptable salt thereof.

23. The shear-activated nanotherapeutic according to any one of claims 20 to 22, wherein the SA-NT is configured to release its constituent nanoparticles at a shear stress of greater than about 100 dynes/cm².

24. The shear-activated nanotherapeutic for use according to any one of claims 20 to 23, wherein the aggregate further comprises nimodipine.

25. The shear-activated nanotherapeutic according to any one of claims 20 to 24, wherein the nanoparticles comprise copolymers of polylactic acid and polyglycolic acid.
